(19) United States
(12) Patent Application Publication
(10) Pub. No.: US 2010/0305427 A1
(43) Pub. Date: Dec. 2, 2010

(54) LONG-RANGE PLANAR SENSOR ARRAY FOR USE IN A SURGICAL NAVIGATION SYSTEM

(75) Inventors: William Hullinger Huber, Scotia, NY (US); Cynthia Elizabeth Landberg Davis, Niskayuna, NY (US); Vernon Thomas Jensen, Draper, UT (US)

Correspondence Address: GE Healthcare, IP Department, 20225 Water Tower Blvd., Mail Code W492, BROOKFIELD, WI, 53045, US

(73) Assignee: General Electric Company, Schenectady, NY (US)

(21) Appl. No.: 12/475,969

(22) Filed: Jun. 1, 2009

(51) Int. Cl. A61B 5/05

(52) U.S. Cl. 600/424

(57) ABSTRACT

A planar sensor array for use in a surgical navigation system comprising at least one substrate and at two layers of a plurality of planar sensor coils formed on or within the at least one insulating substrate. The planar sensor array may be an electromagnetic planar transmitter coil array or an electromagnetic planar receiver coil array that includes at two layers of a plurality of planar electromagnetic transmitter or receiver spiral-shaped coils formed on or within the at least one substrate. The surgical navigation system includes the use of at least one magnetoresistance reference sensor attached to a fixed object; at least one magnetoresistance sensor attached to an object being tracked; and a planar sensor coil array communicating with the at least one magnetoresistance reference sensor and the at least one magnetoresistance sensor to determine the position and orientation of the object being tracked.

### BACKGROUND OF THE INVENTION

This disclosure relates generally to magnetic sensor arrays for position and orientation determination, and more particularly to long-range magnetic planar sensor arrays for use in surgical navigation systems for determining the position and orientation of an object.

Surgical navigation systems track the precise position and orientation of surgical instruments, implants or other medical devices in relation to multidimensional images of a patient's anatomy. Additionally, surgical navigation systems use visualization tools to provide the surgeon with co-registered views of these surgical instruments, implants or other medical devices with the patient's anatomy.

The multidimensional images may be generated either prior to (pre-operative) or during (intraoperative) the surgical procedure. For example, any suitable medical imaging technique, such as x-ray, computed tomography (CT), magnetic resonance (MR), positron emission tomography (PET), ultrasound, or any other suitable imaging technique, as well as any combinations thereof may be utilized. After registering the multidimensional images to the position and orientation of the patient, or to the position and orientation of an anatomical feature or region of interest, the combination of the multidimensional images with graphical representations of the navigated surgical instruments, implants or other medical devices provides position and orientation information that allows a medical practitioner to manipulate the surgical instruments, implants or other medical devices to desired positions and orientations.

Current surgical navigation systems include position and orientation sensors, or sensing sub-systems based on electromagnetic, radio frequency, optical (line-of-sight), and/or mechanical technologies. Surgical navigation systems using these various technologies are used today with limited acceptance in various clinical applications where an x-ray compatible medical table is used. The navigation area is determined by the proximity of the navigation sensors relative to the position of the patient, medical devices and imaging apparatus. A major reason for the limited acceptance of surgical navigation during medical procedures is related to changes required in the normal surgical workflow that complicates the set-up, execution and turn-around time in the operating room. Most navigation enabled medical devices and environments also add mechanical and visual obstructions within the surgical region of interest and the imaging field of view.

These navigation system sensors are typically not radiolucent, and if left in the imaging field of view will cause unwanted x-ray image artifacts. This is true with radiographic imaging, but it is of greater concern with intraoperative fluoroscopic two-dimensional (2D) and three-dimensional (3d) imaging. Based on common constraints across various navigation clinical applications, where intraoperative x-ray imaging is used, the most important region of interest for the navigation system is shared with the most important region of interest for the imaging system. Obvious preferred locations for sensors are not only in the imaging region of interest, but include the area above, below and even within the medical table itself.

Current electromagnetic sensors used in surgical navigation systems typically utilize 3D coils fabricated by winding multiple turns of wire onto bobbins to transmit and receive magnetic fields. To increase the magnetic field strength and range of the electromagnetic sensors at a given power level, the coil size must be increased. However large 3D coils are bulky, expensive to manufacture, and can potentially interfere with the medical procedure workflow. One potential solution is to switch from a bobbin-based 3D coils to planar 2D coils. By utilizing 2D coils, the magnetic field strength of the electromagnetic sensors can be increased without the bulk, cost and workflow shortcomings of larger 3D coils.

Planar 2D electromagnetic coils are typically fabricated into planar 2D electromagnetic coil arrays using traditional low-cost printed circuit board (PCB) fabrication techniques. These techniques typically utilize subtractive patterning of copper foils laminated on a rigid or flexible substrate with plated through-holes of copper. However, these planar 2D electromagnetic coil arrays can introduce large ‘dead zones’ or regions of space with large position and orientation errors at specific orientations. Thus, the effective range for a prior art planar 2D electromagnetic coil array is typically reduced. In addition, copper strongly absorbs x-ray radiation. Unless the copper is very thin (approximately less than 0.1 mm), significant attenuation of the x-ray beam will occur, resulting in noticeable image artifacts and reduced image quality. Unfortunately however, planar 2D electromagnetic coil arrays with thinner copper produces lower magnetic field strengths, resulting in shorter ranges of the electromagnetic sensors.

Therefore, there is a need for long-range radiolucent sensors integrated into the imaging environment of a surgical navigation system that increase range, reduce position and orientation errors, provide uniform x-ray attenuation within the imaging area, improve x-ray transparency, and reduce or eliminate image artifacts.

### BRIEF DESCRIPTION OF THE INVENTION

In accordance with an aspect of the disclosure, a planar sensor array comprising at least one insulating substrate; and at least two layers of a plurality of planar sensor coils formed on or within the at least one insulating substrate.

In accordance with an aspect of the disclosure, an electromagnetic planar transmitter coil array for use with a surgical navigation system comprising at least one substrate; and at least two layers of a plurality of planar electromagnetic transmitter spiral-shaped coils formed on or within the at least one substrate.

In accordance with an aspect of the disclosure, a surgical navigation system comprising at least one magnetoresistance reference sensor attached to a fixed object; at least one magnetoresistance sensor attached to an object being tracked; a planar sensor array communicating with the at least one magnetoresistance reference sensor and the at least one magnetoresistance sensor, a processor coupled to the at least one magnetoresistance reference sensor, the at least one magnetoresistance sensor, and the planar sensor array; and a user interface coupled to the processor.

Various other features, aspects, and advantages will be made apparent to those skilled in the art from the accompanying drawings and detailed description thereof.

### BRIEF DESCRIPTION OF THE DRAWINGS

FIG. 1 is a schematic diagram of an exemplary embodiment of a surgical navigation system;

FIG. 2 is an enlarged top view of an exemplary embodiment of a magnetoresistance sensor;

FIG. 3 is a top view of an exemplary embodiment of a planar sensor array;

FIG. 4 is a cross-sectional view of the planar sensor array of FIG. 3 taken along line 4-4 of FIG. 3;

FIG. 5 is a cross-sectional view of an exemplary embodiment of a planar sensor array;

FIG. 6 is a top view of an exemplary embodiment of a planar sensor array;

FIG. 7 is a top view of an exemplary embodiment of a planar sensor array;

FIG. 8 is a top view of an exemplary embodiment of a planar sensor array;

FIG. 9 is a top view of an exemplary embodiment of a planar sensor array;

FIG. 10 is a top view schematic diagram of an exemplary embodiment of a planar sensor array embedded within a medical table; and

FIG. 11 is a top view schematic diagram of an exemplary embodiment of a planar sensor array embedded within a surgical drape.

### DETAILED DESCRIPTION OF THE INVENTION

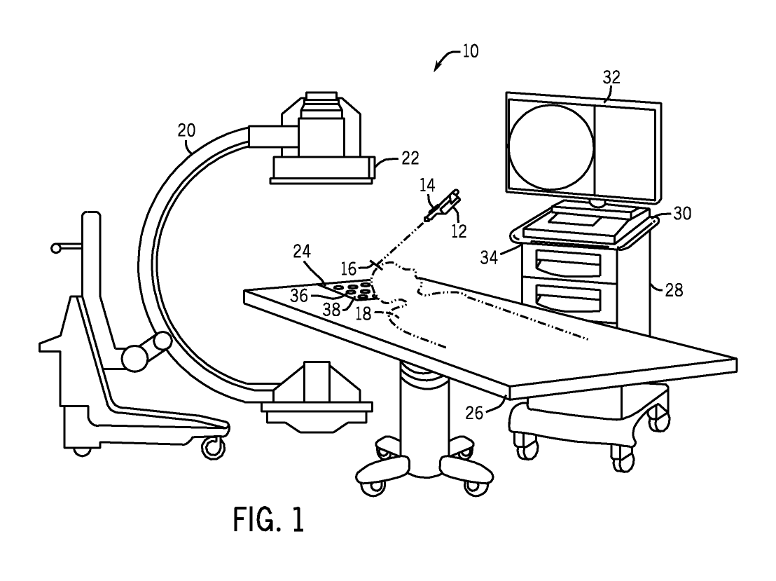

Referring now to the drawings, FIG. 1 illustrates a schematic diagram of an exemplary embodiment of a surgical navigation system 10. The surgical navigation system 10 includes at least one magnetoresistance sensor 12 attached to at least one device 14, at least one magnetoresistance reference sensor 16 rigidly attached to an anatomical reference of a patient 18 undergoing a medical procedure, a planar sensor array 24 positioned on a table 26 supporting the patient 18, and a portable workstation 28. In an exemplary embodiment, the surgical navigation system 10 may also include an imaging apparatus 20 for performing real time imaging during the medical procedure. In an exemplary embodiment, the imaging apparatus 20 may be a mobile fluoroscopic imaging apparatus. In an exemplary embodiment, at least one magnetoresistance reference sensor 22 may be attached to an imaging apparatus 20. In an exemplary embodiment, the portable workstation 28 may include a computer 30, at least one display 32, and a navigation interface 34. The surgical navigation system 10 is configured to operate with the at least one magnetoresistance sensor 12, the magnetoresistance reference sensors 16, 22, and the planar sensor array 24 to determine the position and orientation of the at least one device 14. In an exemplary embodiment, the planar sensor array 24 is radiolucent.

The at least one magnetoresistance sensor 12, the magnetoresistance reference sensors 16, 22 and the planar sensor array 24 are coupled to the navigation interface 34. The at least one magnetoresistance sensor 12, the magnetoresistance reference sensors 16, 22 and the planar sensor array 24 may be coupled to and communicate with the navigation interface 34 through either a wired or wireless connection. The navigation interface 34 is coupled to the computer 30.

The at least one magnetoresistance sensor 12 communicates with and transrmits/receives data from the magnetoresistance reference sensors 16, 22, and the planar sensor array 24. The navigation interface 34 is coupled to and receives data from the at least one magnetoresistance sensor 12, communicates with and transmits/receives data from the magnetoresistance reference sensors 16, 22, and the planar sensor array 24. The surgical navigation system 10 provides the ability to track and display the position and orientation of the at least one device 14 having at least one magnetoresistance sensor 12 attached thereto.

In an exemplary embodiment, the at least one magnetoresistance sensor 12 and the magnetoresistance reference sensors 16, 22 may be configured as magnetic field receivers, and the planar sensor array 24 may be configured as a magnetic field transmitter (generator) for creating at least one magnetic field around the table 26 and the patient 18. The at least one device 14 may be moved relative to the magnetoresistance reference sensors 16, 22 and the planar sensor array 24 within the volume of the at least one magnetic field. In this embodiment, the planar sensor array 24 generates at least one magnetic field that is detected by the at least one magnetoresistance sensor 12 and the magnetoresistance reference sensors 16, 22 resulting in magnetic field measurements.

Theses magnetic field measurements may be used to calculate the position and orientation of the at least one device 14 according to any suitable method or system. For example, the magnetic field measurements are digitized using electronics coupled to the at least one magnetoresistance sensor 12 or the magnetoresistance reference sensors 16, 22, and the digitized signals are transmitted from the at least one magnetoresistance sensor 12 or the magnetoresistance reference sensors 16, 22 to the navigation interface 34. The digitized signals may be transmitted from the at least one magnetoresistance sensor 12 or the magnetoresistance reference sensors 16, 22 to the navigation interface 34 using wired or wireless communication protocols and interfaces. The digitized signals received by the navigation interface 34 represent magnetic information detected by the at least one magnetoresistance sensor 12 or the magnetoresistance reference sensors 16, 22.

The navigation interface 34 transfers the digitized signals to the computer 30 to calculate position and orientation information of the at least one device 14 based on the received digitized signals. The position and orientation information includes the six dimensions (x, y, z, roll, pitch, yaw) for locating the position and orientation of the at least one device 14. This position and orientation information may be transmitted from the computer 30 to the display 32 for review by a medical practitioner.

In an exemplary embodiment, the planar sensor array 24 may be a planar transmitter coil array that includes a plurality of transmitter coils 36 formed on or within a substrate 38. The plurality of transmitter coils 36 may be made of a conductive material. The substrate 38 may be made of an insulating material that is rigid or flexible. In an exemplary embodiment, the sensor array 24 may be incorporated into the table 26, a table mat, or a surgical drape.

In an exemplary embodiment, the at least one magnetoresistance sensor 12 and the magnetoresistance reference sensors 16, 22 may be configured as magnetic field transmitters (generators), and the planar sensor array 24 may be configured as a magnetic field receiver. In this embodiment, the at least one magnetoresistance sensor 12 and the magnetoresistance reference sensors 16, 22 generate magnetic fields having different frequencies that are detected by the planar sensor array 24 resulting in magnetic field measurements.

In an exemplary embodiment, the at least one magnetoresistance sensor 12 and the magnetoresistance reference sensors 16, 22, and the planar sensor array 24 may be powered by a battery or batteries, or through inductive coupling.

In an exemplary embodiment, the at least one magnetoresistance sensor 12 and the magnetoresistance reference sensors 16, 22, and the planar sensor array 24 may include power conversion and drive circuitry for energizing the sensors,

In an exemplary embodiment, the at least one magnetoresistance sensor 12 and the magnetoresistance reference sensors 16, 22, and the planar sensor array 24 may include storage and processing circuitry for storing and processing data.

In an exemplary embodiment, the at least one magnetoresistance sensor 12 and the magnetoresistance reference sensors 16, 22, and the planar sensor array 24 may include bi-directional wireless communication circuitry and protocols for transmitting and receiving data.

In an exemplary embodiment, the planar sensor array 24 may be an induction power source for the at least one magnetoresistance sensor 12 and the magnetoresistance reference sensors 16, 22 that may be configured as wireless transmitters.

The surgical navigation system 10 described herein is capable of tracking many different types of devices 14 during different procedures. Depending on the procedure, the at least one device 14 may be a surgical instrument (e.g., an imaging catheter, a diagnostic catheter, a therapeutic catheter, a guide wire, a debrider, an aspirator, a handle, a guide, etc.), a surgical implant (e.g., an artificial disk, a bone screw, a shunt, a pedicle screw, a plate, an intramedullary rod, etc.), or some other device. Depending on the context of the usage of the surgical navigation system 10, any number of suitable devices 14 may be used. In an exemplary embodiment, there may be more than one device 14, and more than one magnetoresistance sensor 12 attached to each device 14.

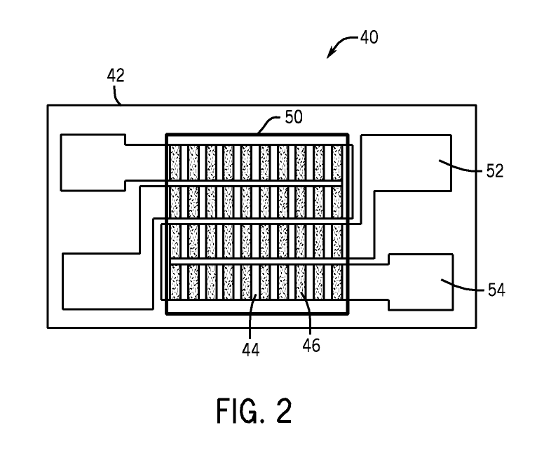

FIG. 2 illustrates an enlarged top view of an exemplary embodiment of a magnetoresistance sensor 40. The magnetoresistance sensor 40 may be implemented as the at least one magnetoresistance sensor 12 or the magnetoresistance reference sensors 16, 22 shown in FIG. 1. The magnetoresistance sensor 40 provides a change in electrical resistance of a conductor or semiconductor when a magnetic field is applied. The sensor's resistance depends upon the magnetic field applied. As shown in FIG. 2, the a magnetoresistance sensor 40 comprises an insulating substrate 42, an alternating pattern of a metal material 44 and a semiconductor material 46 deposited on a surface 48 of the insulating substrate, and a bias magnet material 50 deposited over the alternating pattern of metal material 44 and semiconductor material 46. The alternating pattern of metal material 44 and semiconductor material 46 creates a composite structure with alternating bands of metal material 44 and semiconductor material 46. At least one input connection contact 52 is coupled to the metal material 44 and at least one output connection contact 54 is coupled to the metal material 44. The magnetoresistance sensor 40 is radiolucent.

The semiconductor material 46 may be series connected to increase the magnetoresistance sensor 40 resistance. In an exemplary embodiment, the semiconductor material 46 may be comprised of a single semiconductor element. The bias magnet material 50 subjects the semiconductor material 46 to a magnetic field required to achieve required sensitivity. The magnetoresistance sensor 40 provides a signal in response to the strength and direction of a magnetic field. In an exemplary embodiment, the magnetic field may be approximately 0.1 to 0.2 Tesla.

The application of a magnetic field confines the electrons to the semiconductor material 46, resulting in an increased path length. Increasing the path length, increases the sensitivity of the magnetoresistance sensor 40. The magnetic field also increases the resistance of the magnetoresistance sensor 40. In the geometry disclosed in FIG. 2, at a zero magnetic field, the current density is uniform throughout the magnetoresistance sensor 40. At a high magnetic field, the electrons (or holes) propagate radially outward toward the corners of the semiconductor material 46, resulting in a large magnetoresistance (high resistance).

Many new clinical applications include tracking of a variety of devices including catheters, guide wires, and other endovascular instruments that require sensors to be very small in size (millimeter dimensions or smaller). The form factor of the magnetoresistance sensor 40 may be scaled to sizes less than 0.1 mm×0.15 mm.

In an exemplary embodiment, the magnetoresistance sensor may be built with various architectures and geometries, including, giant magnetoresistance (GMR) sensors, and extraordinary magnetoresistance (EMR) sensors.

The magnetoresistance sensor 40 provides a very small form factor, excellent signal-to-noise ratio (low noise operation), and excellent low frequency response. Low noise combined with wide dynamic range enables the magnetoresistance sensor 40 to be used for position and orientation tracking in surgical navigation systems. The low frequency response of the magnetoresistance sensor 40 allows a surgical navigation system to operate at very low frequencies where metal tolerance is maximized.

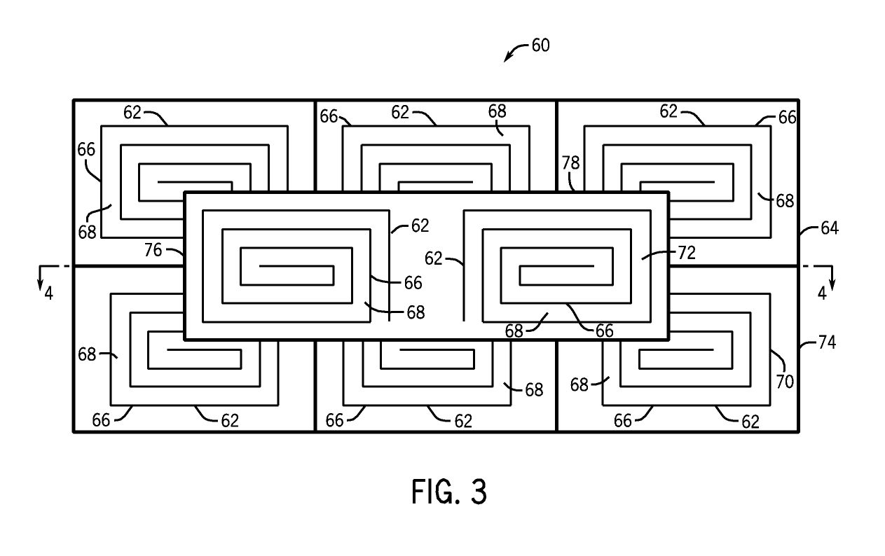

FIG. 3 illustrates a top view of an exemplary embodiment of a planar sensor array 60. The planar sensor array 60 may be implemented as the planar sensor array 24 shown in FIG. 1. The planar sensor array 60 may be an electromagnetic planar transmitter or receiver coil array. It is well known by the electromagnetic principle of reciprocity, that a description of a coil's properties as a transmitter may also be used to understand the coil's properties as a receiver. Therefore, the planar sensor array 60 may be used as a transmitter or a receiver.

The planar sensor array 60 includes a plurality of planar sensor coils 62 formed on or within at least one substrate 64 and arranged in a specific configuration to eliminate dead zones. The plurality of planar sensor coils 62 may be made of a conductive material forming a plurality of conductor traces 66 with spaces 68 in-between. The at least one substrate 64 may be made of an insulating material that is rigid or flexible. In an exemplary embodiment, the planar sensor array 60 includes at least two layers 70, 72 of a plurality of planar sensor coils 62 formed on or within at least one substrate 64. A first layer 70 of a plurality of planar sensor coils 62 is arranged on or within a first layer 74 of the at least one substrate 64. A second layer 72 of a plurality of planar sensor coils 62 is arranged on or within a second layer 76 of the at least one substrate 64. The second layer 72 of the plurality of planar sensor coils 62 is positioned above the first layer 70 of the plurality of planar sensor coils 62, and overlaps a center portion 78 of the first layer 70 of the plurality of planar sensor coils 62.

In the exemplary embodiment shown in FIG. 3, the planar sensor array 60 includes the first layer 70 of at least six rectangular-shaped spiral coils 62, arranged on or within the first layer 74 of the at least one substrate 64 in a 3×2 pattern, and the second layer 72 of at least two rectangular-shaped spiral coils 62 arranged on or within the second layer 76 of the at least one substrate 64.

In an exemplary embodiment, the planar sensor array 60 may be fabricated using a printed circuit board (PCB) fabrication technique. This technique may utilize subtractive patterning of conductor traces 66 laminated or etched on a rigid or a flexible substrate 64. In an exemplary embodiment, the rigid substrate may be fabricated from a Flame Retardant 4 (FR-4) PCB substrate material. In an exemplary embodiment, the flexible substrate may be fabricated from a polyimide PCB substrate material. In an exemplary embodiment, the conductor traces 66 may be made of a conductive, low density, low resistivity, and radiolucent material. This material may include aluminum, magnesium, carbon nanotubes, graphene, titanium, and their various alloys. This material enables x-ray transparency, minimizes power dissipation, and is lightweight. In an exemplary embodiment, the planar sensor array 60 is x-ray transmissive over its entire area.

In an exemplary embodiment, the planar sensor array 60 may be an electromagnetic planar transmitter coil array that includes a plurality of planar electromagnetic transmitter spiral-shaped coils 62 formed on or within at least one substrate 64 and arranged in a specific configuration to eliminate dead zones. The plurality of planar electromagnetic transmitter spiral-shaped coils 62 may be made of a conductive material forming a plurality of conductor traces 66 with spaces 68 in-between. The substrate 64 may be made of an insulating material that is rigid or flexible. The plurality of planar electromagnetic transmitter spiral-shaped coils 62 may be arranged to generate electromagnetic fields and gradients in all three Cartesian coordinate axis (x, y, and z) directions and provide for position and orientation measurements of at least one device having at least one magnetoresistance sensor attached thereto including all six position and orientation degrees of freedom coordinates including x, y, z, roll, pitch, and yaw.

In an exemplary embodiment, the planar sensor array 60 may be an electromagnetic planar receiver coil array that includes a plurality of planar electromagnetic receiver spiral-shaped coils 62 formed on or within at least one substrate 64 and arranged in a specific configuration to eliminate dead zones. The plurality of planar electromagnetic receiver spiral-shaped coils 62 may be made of a conductive material forming a plurality of conductor traces 66 with spaces 68 in-between. The substrate 64 may be made of an insulating material that is rigid or flexible.

In an exemplary embodiment, the planar sensor array 60 may be switchable between an electromagnetic planar receiver coil array and an electromagnetic planar transmitter coil array. In this embodiment, the planar sensor array 60 may include additional electronic circuitry for switching the planar sensor array 60 functionality between a receiver and a transmitter as needed, or perhaps alternate continuously at various duty cycles as needed for specific clinical applications.

In an exemplary embodiment, the planar sensor array 60 may include spiral-shaped coils 62 with curved conductor traces 66 or straight conductor traces 66.

In an exemplary embodiment, the planar array 60 may include a plurality of radiopaque fiducial markers for image verification and calibration.

In an exemplary embodiment, the planar sensor array 60 may be incorporated into a medical table, a table mat, or a surgical drape. In an exemplary embodiment, the planar sensor array 60 may be integrated into an imaging apparatus near the x-ray source or near the x-ray detector. In an exemplary embodiment, the planar sensor array 60 may be integrated into other attachable devices that are located in the active image area during x-ray imaging, such as for example, laser aiming devices, distortion correction devices, image chain modeling devices, alignment targets, navigation targets, etc.

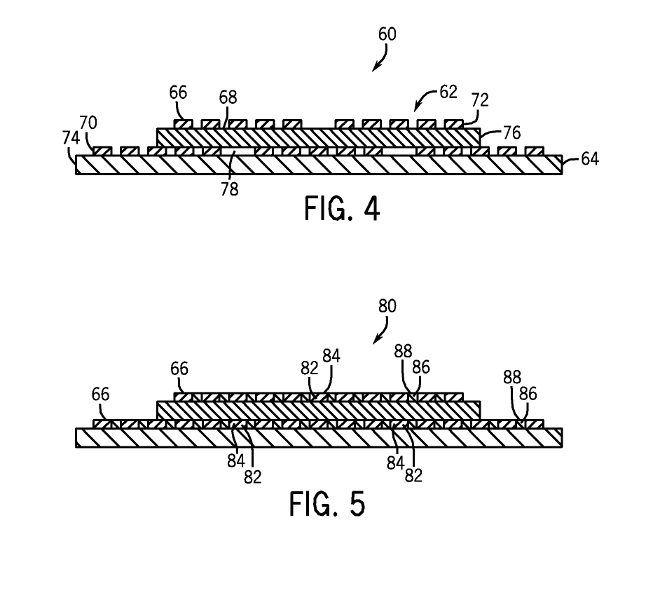

FIG. 4 illustrates a cross-sectional view of the planar sensor array 60 of FIG. 3. The planar sensor array 60 includes at least two layers 70, 72 of a plurality of planar sensor coils 62 formed on or within at least one substrate 64. A first layer 70 of a plurality of planar sensor coils 62 is arranged on or within a first layer 74 of the at least one substrate 64. A second layer 72 of a plurality of planar sensor coils 62 is arranged on or within a second layer 76 of the at least one substrate 64. The second layer 72 of the plurality of planar sensor coils 62 is positioned above the first layer 70 of the plurality of planar sensor coils 62, and overlaps a center portion 78 of the first layer 70 of the plurality of planar sensor coils 62.

FIG. 5 illustrates a cross-sectional view of an exemplary embodiment of a planar sensor array 80. The planar sensor array 80 includes at least two layers 70, 72 of a plurality of planar sensor coils 62 formed on or within at least one substrate 64. A first layer 70 of a plurality of planar sensor coils 62 is arranged on or within a first layer 74 of the at least one substrate 64. A second layer 72 of a plurality of planar sensor coils 62 is arranged on or within a second layer 76 of the at least one substrate 64. The second layer 72 of the plurality of planar sensor coils 62 is positioned above the first layer 70 of the plurality of planar sensor coils 62, and overlaps a center portion 78 of the first layer 70 of the plurality of planar sensor coils 62.

In an exemplary embodiment, the planar sensor array 80 may include a conductive radiolucent material 82 filling the open areas 84 within the non-coil regions of the planar sensor array 80. The conductive radiolucent material may include aluminum, magnesium, carbon nanotubes, graphene, titanium, and their various alloys.

In an exemplary embodiment, the planar sensor array 80 may include an epoxy or other similar material 86 having an x-ray density approximately matched to the conductor trace material 66 filling open areas 88 in-between the conductor traces 66 of the planar sensor array 80.

By having conductor traces made of a conductive, low density, low resistivity, radiolucent material; filing the open areas 84 within the non-coil regions of the planar sensor array 80 with conductive radiolucent material 82; and filing the open areas 88 in-between, the conductor traces 66 with an epoxy or other similar material 86 that is approximately x-ray density matched to the conductor trace material 66 the planar sensor array 80 appears as x-ray transmissive over its entire area and thus minimizes image artifacts. The coil design is optimized to increase the range of the planar sensor array 80 reduce dead zones in the planar sensor array 80, and reduce artifacts in x-ray images.

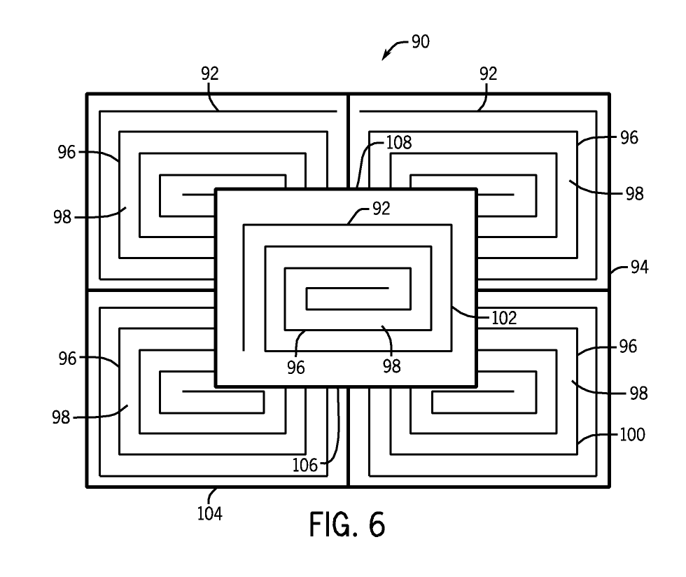

FIG. 6 illustrates a top view of an exemplary embodiment of a planar sensor array 90. The planar sensor array 90 may be implemented as the planar sensor array 24 shown in FIG. 1. The planar sensor array 90 may be an electromagnetic planar transmitter coil array or an electromagnetic planar receiver coil array.

The planar sensor array 90 includes a plurality of planar sensor coils 92 formed on or within at least one substrate 94 and arranged in a specific configuration to eliminate dead zones. The plurality of planar sensor coils 92 may be made of a conductive material forming a plurality of conductor traces 96 with spaces 98 in-between. The at least one substrate 94 may be made of an insulating material that is rigid or flexible. In an exemplary embodiment, the planar sensor array 90 includes at least two layers 100, 102 of a plurality of planar sensor coils 92 formed on or within at least one substrate 94. A first layer 100 of a plurality of planar sensor coils 92 is arranged on or within a first layer 104 of the at least one substrate 94. A second layer 102 of a plurality of planar sensor coils 92 is arranged on or within a second layer 106 of the at least one substrate 94. The second layer 102 of the plurality of planar sensor coils 92 is positioned above the first layer 100 of the plurality of planar sensor coils 92, and overlaps a center portion 108 of the first layer 100 of the plurality of planar sensor coils 92.

In the exemplary embodiment shown in FIG. 6, the planar sensor array 90 includes the first layer 100 of at least four rectangular-shaped spiral coils 92 arranged on or within the first layer 104 of the at least one substrate 94 in a 2×2 pattern, and the second layer 102 of at least one rectangular-shaped spiral coil 92 arranged on or within the second layer 106 of the at least one substrate 94. The plurality of rectangular-shaped spiral coils 92 are arranged to venerate electromagnetic fields and gradients in all three Cartesian coordinate axis (x, y, and z) directions and provide for position and orientation measurements of at least one device having at least one magnetoresistance sensor attached thereto including all six position and orientation degrees of freedom coordinates including x, y, z, roll, pitch, and yaw.

In an exemplary embodiment, the planar sensor array 90 may be fabricated using a PCB fabrication technique. This technique may utilize subtractive patterning of conductor traces 96 laminated or etched on a rigid or a flexible substrate 94. In an exemplary embodiment, the rigid substrate may be fabricated from a FR4 PCB substrate material. In an exemplary embodiment, the flexible substrate may be fabricated from a polyimide PCB substrate material. In an exemplary embodiment, the conductor traces 96 may be made of a conductive, low density, low resistivity, radiolucent material. This material may include aluminum, magnesium, carbon nanotubes, graphene, titanium, and their various alloys. This material enables x-ray transparency, minimizes power dissipation, and is lightweight. In an exemplary embodiment, the planar sensor array 90 is x-ray transmissive over its entire area.

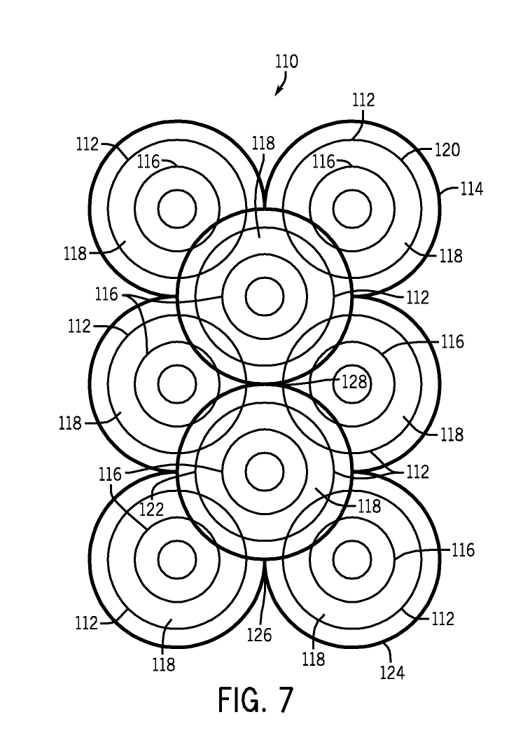

FIG. 7 illustrates a top view of an exemplary embodiment of a planar sensor array 110. The planar sensor array 110 may be implemented as the planar sensor array 24 shown in FIG. 1. The planar sensor array 110 may be an electromagnetic planar transmitter coil array or an electromagnetic planar receiver coil array.

The planar sensor array 110 includes a plurality of planar sensor coils 112 formed on or within at least one substrate 114 and arranged in a specific configuration to eliminate dead zones. The plurality of planar sensor coils 112 may be made of a conductive material forming a plurality of conductor traces 116 with spaces 118 in-between. The at least one substrate 114 may be made of an insulating material that is rigid or flexible. In an exemplary embodiment, the planar sensor array 110 includes at least two layers 120, 122 of a plurality of planar sensor coils 112 formed on or within at least one substrate 114. A first layer 120 of a plurality of planar sensor coils 112 is arranged on or within a first layer 124 of the at least one substrate 114. A second layer 122 of a plurality of planar sensor coils 112 is arranged on or within a second layer 126 of the at least one substrate 114. The second layer 122 of the plurality of planar sensor coils 112 is positioned above the first layer 120 of the plurality of planar sensor coils 112, and overlaps a center portion 128 of the first layer 120 of the plurality of planar sensor coils 12.

In the exemplary embodiment shown in FIG. 7, the planar sensor array 110 includes the first layer 120 of at least six circular or elliptical shaped spiral coils 112 arranged on or within the first layer 124 of the at least one substrate 114 in a 3×2 pattern, and the second layer 122 of at least two circular or elliptical shaped spiral coils 112 arranged on or within the second layer 126 of the at least one substrate 114. The plurality of rectangular-shaped spiral coils 112 are arranged to generate electromagnetic fields and gradients in all three Cartesian coordinate axis (x, y, and z) directions and provide for position and orientation measurements of at least one device having at least one magnetoresistance sensor attached thereto including all six position and orientation degrees of freedom coordinates including x, y, z, roll, pitch, and yaw.

In an exemplary embodiment, the planar sensor array 113 may be fabricated using a PCB fabrication technique. This technique may utilize subtractive patterning of conductor traces 116 laminated or etched on a rigid or a flexible substrate 114, in an exemplary embodiment, the rigid substrate may be fabricated from a FR4 PCB substrate material. In an exemplary embodiment, the flexible substrate may be fabricated from a polyimide PCB substrate material. In an exemplary embodiment, the conductor traces 116 may be made of a conductive, low density, low resistivity, radiolucent material. This material may include aluminum, magnesium, carbon nanotubes, graphene, titanium, and their various alloys. This material enables x-ray transparency, minimizes power dissipation, and is lightweight. In an exemplary embodiment, the planar sensor array 110 is x-ray transmissive over its entire area.

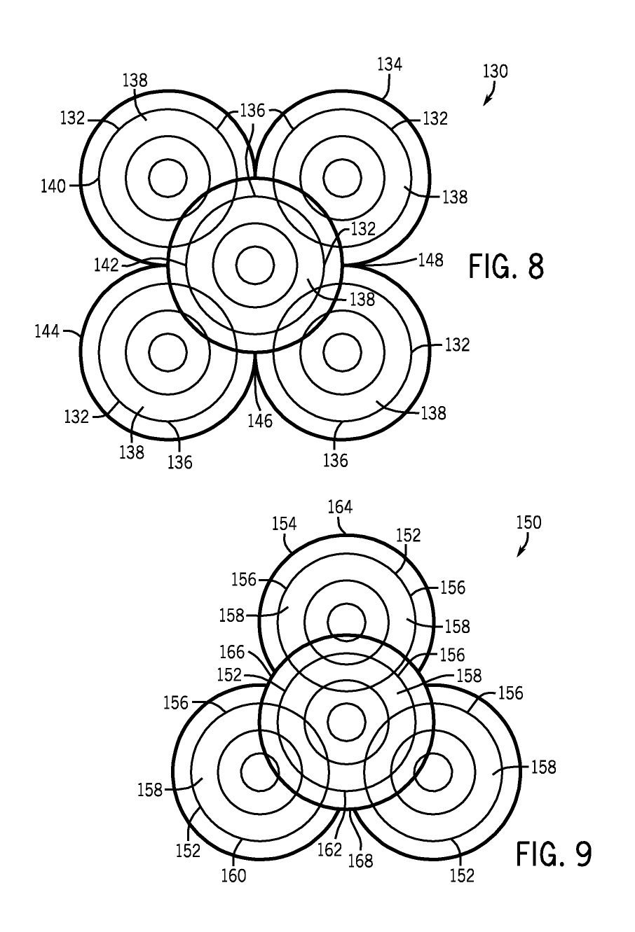

FIG. 8 illustrates a top view of an exemplary embodiment of a planar sensor array 130. The planar sensor array 130 may be implemented as the planar sensor array 24 shown in FIG. 1. The planar sensor array 130 may be an electromagnetic planar transmitter coil array or an electromagnetic planar receiver coil array.

The planar sensor array 130 includes a plurality of planar sensor coils 132 formed on or within at least one substrate 134 and arranged in a specific configuration to eliminate dead zones. The plurality of planar sensor coils 132 may be made of a conductive material forming a plurality of conductor traces 136 with spaces 138 in-between. The at least one substrate 134 may be made of an insulating material that is rigid or flexible. In an exemplary embodiment, the planar sensor array 130 includes at least two layers 140, 142 of a plurality of planar sensor coils 132 formed on or within at least one substrate 134. A first layer 140 of a plurality of planar sensor coils 132 is arranged on or within a first layer 144 of the at least one substrate 134. A second layer 142 of a plurality of planar sensor coils 132 is arranged on or within a second layer 146 of the at least one substrate 134. The second layer 142 of the plurality of planar sensor coils 132 is positioned above the first layer 140 of the plurality of planar sensor coils 132, and overlaps a center portion 148 of the first layer 140 of the plurality of planar sensor coils 132.

In the exemplary embodiment shown in FIG. 3, the planar sensor array 130 includes the first layer 140 of at least six circular or elliptical shaped spiral coils 132 arranged on or within the first layer 144 of the at least one substrate 134 in a 2×2 pattern, and the second layer 142 of at least one circular or elliptical shaped spiral coil 132 arranged on or within the second layer 146 of the at least one substrate 134. The plurality of rectangular-shaped spiral coils 132 are arranged to generate electromagnetic fields and gradients in all three Cartesian coordinate axis (x, y, and z) directions and provide for position and orientation measurements of at least one device having at least one magnetoresistance sensor attached thereto including all six position and orientation degrees of freedom coordinates including x, y, z, roll, pitch, and yaw.

In an exemplary embodiment, the planar sensor array 130 may be fabricated using a PCB fabrication technique. This technique may utilize subtractive patterning of conductor traces 136 laminated or etched on a rigid or a flexible substrate 134. In an exemplary embodiment, the rigid substrate may be fabricated from a FR4 PCB substrate material. In an exemplary embodiment, the flexible substrate may be fabricated from a polyimide PCB substrate material. In an exemplary embodiment, the conductor traces 136 may be made of a conductive, low density, low resistivity, radiolucent material. This material may include aluminum, magnesium, carbon nanotubes, graphene, titanium, and their various alloys. This material enables x-ray transparency, minimizes power dissipation, and is lightweight. In an exemplary embodiment, the planar sensor array 130 is x-ray transmissive over its entire area.

FIG. 9 illustrates a top view of an exemplary embodiment of a planar sensor array 150. The planar sensor array 150 may be implemented as the planar sensor array 24 shown in FIG. 1. The planar sensor array 150 may be an electromagnetic planar transmitter coil array or an electromagnetic planar receiver coil array.

The planar sensor array 150 includes a plurality of planar sensor coils 152 formed on or within at least one substrate 154 and arranged in a specific configuration to eliminate dead zones. The plurality of planar sensor coils 152 may be made of a conductive material forming a plurality of conductor traces 156 with spaces 158 in-between. The at least one substrate 154 may be made of an insulating material that is rigid or flexible. In an exemplary embodiment, the planar sensor array 150 includes at least two layers 160, 162 of a plurality of planar sensor coils 152 formed on or within at least one substrate 154. A first layer 160 of a plurality of planar sensor coils 152 is arranged on or within a first layer 164 of the at least one substrate 154. A second layer 162 of a plurality of planar sensor coils 152 is arranged on or within a second layer 166 of the at least one substrate 154. The second layer 162 of the plurality of planar sensor coils 152 is positioned above the first layer 160 of the plurality of planar sensor coils 152, and overlaps a center portion 168 of the first layer 160 of the plurality of planar sensor coils 152.

In the exemplary embodiment shown in FIG. 8, the planar sensor array 150 includes the first layer 160 of at least six circular or elliptical shaped spiral coils 152 arranged on or within the first layer 164 of the at least one substrate 154 in a 2×1 pattern, and the second layer 162 of at least one circular or elliptical shaped spiral coil 152 arranged on or within the second layer 166 of the at least one substrate 154. The plurality of rectangular-shaped spiral coils 152 are arranged to generate electromagnetic fields and gradients in all three Cartesian coordinate axis (x, y, and z) directions and provide for position and orientation measurements of at least one device having at least one magnetoresistance sensor attached thereto including all six position and orientation degrees of freedom coordinates including x, y, z, roll, pitch, and yaw.

In an exemplary embodiment, the planar sensor array 150 may be fabricated using a PCB fabrication technique. This technique may utilize subtractive patterning of conductor traces 156 laminated or etched on a rigid or a flexible substrate 154. In an exemplary embodiment, the rigid substrate may be fabricated from a FR4 PCB substrate material. In an exemplary embodiment, the flexible substrate may be fabricated from a polyimide PCB substrate material. In an exemplary embodiment, the conductor traces 156 may be made of a conductive, low density, low resistivity, radiolucent material. This material may include aluminum, magnesium, carbon nanotubes, graphene, titanium, and their various alloys. This material enables x-ray transparency, minimizes power dissipation, and is lightweight. In an exemplary embodiment, the planar sensor array 150 is x-ray transmissive over its entire area.

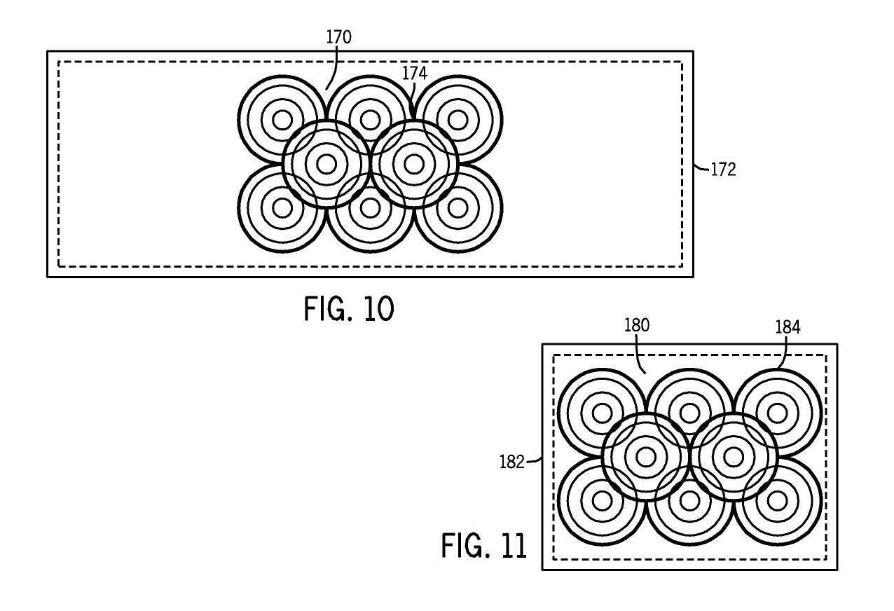

FIG. 10 illustrates a top view schematic diagram of an exemplary embodiment of a planar sensor array 170 embedded within a medical table 172. The medical table 172 may be used with the surgical navigation system 10 of FIG. 1. The planar sensor array 170 includes a plurality of planar sensor coils 174. The planar sensor array 170 may be integrated into the table 172 in any suitable manner. The planar sensor array 170 integrated into the medical table 172 is radiolucent.

As described above, by embedding the planar sensor array 170 into medical table 172, the plurality of planar sensor coils 174 become fixed with respect to the medical table 172. In this way, magnetic field distortions normally caused by medical table 172 may be corrected by creating a magnetic field map at the time the medical table 172 is manufactured. In contrast, by not integrating the planar sensor array 170 into the medical table 172, any magnetic field distortion caused by the medical table 172 must either be accounted for by creating a distortion-free table or by mapping the magnetic field before each and every use.

In an exemplary embodiment, the medical table 172 may include, for example, an operating room table, an x-ray imaging table, a combination operating and imaging table, or a Jackson table, generally used for spine and orthopedic applications. In addition, medical table 172 may include any other medical apparatus that could benefit from tracking technology, including, for example, an imaging apparatus useful in x-ray examinations of patients.

FIG. 11 illustrates a top view schematic diagram of an exemplary embodiment of a planar sensor array 180 embedded within a surgical drape 182. The surgical drape 182 may be used with the surgical navigation system 10 of FIG. 1. The planar sensor array 180 includes a plurality of planar sensor coils 184. The planar sensor array 180 may be integrated into the surgical drape 182 in any suitable manner. The surgical drape 182 may be placed over a table or over a patient during a medical procedure. The surgical drape 182 includes a planar sensor array 180 integrated therein. The surgical drape 182 may be a single use or multiple use disposable product. The planar sensor array 180 integrated into the surgical drape 182 is radiolucent.

In an exemplary embodiment, a planar sensor array may be integrated into a table, table mat, or surgical drape of a surgical navigation system for improving surgical navigation workflow and eliminating image artifacts from intraoperative images. In an exemplary embodiment, the planar sensor array may be located within a table, table mat, surgical drape, or adjacent to a table surface.

While the disclosure has been described with reference to various embodiments, those skilled in the art will appreciate that certain substitutions, alterations and omissions may be made to the embodiments without departing from the spirit of the disclosure. Accordingly, the foregoing description is meant to be exemplary only, and should not limit the scope of the disclosure as set forth in the following claims.

What is claimed is;

1. A planar sensor array comprising:
at least one insulating substrate; and
at least two layers of a plurality of planar sensor coils formed on or within the at least one insulating substrate.

2. The planar sensor array of claim 1, further comprising a first layer of a plurality of planar sensor coils arranged on or within a first layer of the at least one insulating substrate.

3. The planar sensor array of claim 2, further comprising a second layer of a plurality of planar sensor coils arranged on or within a second layer of the at least one insulating substrate.

4. The planar sensor array of claim 3, wherein the second layer of the plurality of planar sensor coils is positioned above the first layer of the plurality of planar sensor coils, and overlaps a center portion of the first layer of the plurality of planar sensor coils.

5. The planar sensor array of claim 1, wherein the plurality of planar sensor coils are made of a conductive material forming a plurality of conductor traces with spaces in-between.

6. The planar sensor array of claim 5, wherein the conductor traces are made of a conductive, low density, low resistivity, radiolucent material.

7. The planar sensor array of claim 6, wherein the conductive, low density, low resistivity, radiolucent material is selected from the group consisting of aluminum, magnesium, carbon nanotubes, graphene, titanium, and their various alloys.

8. The planar sensor array of claim 1, wherein the plurality of planar sensor coils are rectangular-shaped spiral coils.

9. The planar sensor array of claim 1, wherein the plurality of planar sensor coils are circular or elliptical shaped spiral coils.

10. The planar sensor array of claim 1, wherein the plurality of planar sensor coils are a plurality of planar electromagnetic transmitter spiral-shaped coils formed on or within the at least one insulating substrate.

11. The planar sensor array of claim 1, wherein the plurality of planar sensor coils are a plurality of planar electromagnetic receiver spiral-shaped coils formed on or within the at least one insulating substrate.

12. The planar sensor array of claim 1, further comprising a conductive radiolucent material filling open areas within non-coil regions of the planar sensor array.

13. The planar sensor array of claim 12, wherein the conductive radiolucent material is selected from the group consisting of aluminum, magnesium, carbon nanotubes, graphene, titanium, and their various alloys.

14. The planar sensor array of claim 6, further comprising an epoxy or other similar material having an x-ray density approximately matched to the conductor trace material filling open areas in-between the conductor traces of the planar sensor array.

15. The planar sensor array of claim 1, wherein the at least one insulating substrate is rigid.

16. The planar sensor array of claim 1, wherein the at least one insulating substrate is flexible.

17. The planar sensor array of claim 1, wherein the planar sensor array is incorporated into at least one of a medical table, a table mat, or a surgical drape.

18. The planar sensor array of claim 1, wherein the planar sensor array is switchable between an electromagnetic planar receiver coil array and an electromagnetic planar transmitter coil array.

19. The planar sensor array of claim 18, wherein the planar sensor array includes additional electronic circuitry for switching the planar sensor array functionality between a receiver and a transmitter.

20. The planar sensor array of claim 19, wherein the planar sensor array functionality is switched as needed.

21. The planar sensor array of claim 19, wherein the planar sensor array functionality is switched continuously at various duty cycles.

22. An electromagnetic planar transmitter coil array for use with a surgical navigation system comprising:
at least one substrate; and
at least two layers of a plurality of planar electromagnetic transmitter spiral-shaped coils formed on or within the at least one substrate.

23. The planar sensor array of claim 22, further comprising a first layer of a plurality of planar electromagnetic transmitter spiral-shaped coils arranged on or within a first layer of the at least one substrate.

24. The planar sensor array of claim 23, further comprising a second layer of a plurality of planar electromagnetic transmitter spiral-shaped coils arranged on or within a second layer of the at least one substrate.

25. The planar sensor array of claim 24, wherein the second layer of the plurality of planar electromagnetic transmitter spiral shaped coils is positioned above the first layer of the plurality of planar electromagnetic transmitter spiral-shaped coils, and overlaps a center portion of the first layer of the plurality of planar electromagnetic transmitter spiral-shaped coils.

26. The planar sensor array of claim 22, wherein the plurality of planar electromagnetic transmitter spiral-shaped coils are made of a conductive material forming a plurality of conductor traces with spaces in-between.

27. The planar sensor array of claim 26, wherein the conductor traces are made of a conductive, low density, low resistivity, radiolucent material.

28. The planar sensor array of claim 27, wherein the conductive, low density, low resistivity, radiolucent material is selected from the group consisting of aluminum, magnesium, carbon nanotubes, graphene, titanium, and their various alloys.

29. The planar sensor array of claim 22, further comprising a conductive radiolucent material filling open areas within non-coil regions of the electromagnetic planar transmitter coil array.

30. The planar sensor array of claim 29, wherein the conductive radiolucent material is selected from the group consisting of aluminum, magnesium, carbon nanotubes, graphene, titanium, and their various alloys.

31. The planar sensor array of claim 27, further comprising an epoxy or other similar material having an x-ray density approximately matched to the conductor trace material filling open areas in-between the conductor traces of the electromagnetic planar transmitter coil array.

32. The planar sensor array of claim 22, wherein the electromagnetic planar transmitter coil array is incorporated into at least one of a medical table, a table mat, or a surgical drape.

33. A surgical navigation system comprising:
at least one magnetoresistance reference sensor attached to a fixed object;
at least one magnetoresistance sensor attached to an object being tracked;
a planar sensor array communicating with the at least one magnetoresistance reference sensor and the at least one magnetoresistance sensor;
a processor coupled to the at least one magnetoresistance reference sensor, the at least one magnetoresistance sensor, and the planar sensor array; and
a user interface coupled to the processor.

34. The surgical navigation system of claim 33, wherein the planar sensor array is an electromagnetic planar transmitter coil array that includes a plurality of planar electromagnetic transmitter spiral-shaped coils formed on or within at least one substrate.

35. The surgical navigation system of claim 33, wherein the processor calculates position and orientation data of the object being tracked.

36. The surgical navigation system of claim 35, wherein the user interface provides visualization of the position and orientation data to an operator.
